(19) United States
(12) Patent Application Publication
(10) Pub. No.: US 2003/0171892 A1
(43) Pub. Date: Sep. 11, 2003

(54) Method for identifying a person among a population by sensing his fingerprints

(76) Inventors: Pierre Chastel, Bourron-Marlotte (FR); Jean-Christophe Fondeur, Levalbo (FR)

Correspondence Address: LARSON & TAYLOR, PLC, 1199 NORTH FAIRFAX STREET, SUITE 900, ALEXANDRIA, VA, 22314, US

(21) Appl. No.: 10/311,004

(22) PCT Filed: Jun. 13, 2001
(86) PCT No.: PCT/FR01/01838

(30) Foreign Application Priority Data
Jun. 16, 2000 (FR) 0007707

(51) Int. Cl. G01B 5/20

(52) U.S. Cl. 702/167

(57) ABSTRACT

The invention concerns a method for identifying a person among a population by sensing the fingerprints of at least one finger of his hand and comparing said fingerprints with fingerprint data of all the persons of the population previously stored in a file (1); it consists in sensing in the first place the shape of at least one hand of all the persons and in storing hand shape data in the file (1) in correlation with respective fingerprint data; then during the process identifying a person, in detecting (in 3) the shape of his hand and (in 2) the fingerprints of at least one finger; comparing (5) the data concerning the shape of his hand with the data concerning the hand shapes stored in the file (1) for the whole population; selecting (6) in the file a group consisting of hand shapes substantially identical with the sensed shape of the hand of the person to be identified; and comparing (8) the sensed fingerprints of the person to be identified with the data stored in the file in correlation with respectively the hand shapes of only the persons of the selected group.

The present invention relates to improvements made to the methods for identifying a person among a population, by sensing the fingerprints of at least one finger of the hand of said person and comparing these fingerprints with fingerprint information of all the persons of said population previously stored in a file.

It will firstly be stressed that the invention is essentially concerned with the “identification” of a person taken from a population, that is to say with the comparison of a person's characteristic elements with characteristic elements stored in a file and grouping together the characteristic elements of all the persons of the population, and that the invention does not relate to the “authentication” of a person, that is to say the comparison of the person's characteristic elements with identical characteristic elements held in a file which is queried or that said person holds in duplicate (chip card secret code for example).

It is known to identify a person, with a small risk of error, with the aid of the fingerprints of at least one of his/her fingers of at least one of his/her hands. There are currently various types of devices making it possible to sense fingerprints, and the corresponding information, transformed into the form of digitized electrical signals, may be stored for subsequent comparison and identification purposes.

The comparison of the fingerprints sensed on a finger of a person with the digitized information stored in a file is a relatively complex and lengthy operation. The identification, through this criterion alone, of a person, among a population of several million, or even several tens, or even several hundreds of millions of persons is certainly still achievable, but in practice would require very sizeable computing means.

Certainly, a preselection performed according to other criteria could be performed. For example, a straightforward preselection could be undertaken according to the sex of the persons; however, such a preselection affords approximately only a halving of the information to be processed, which is very inadequate and would not significantly cut the aforesaid cost of the hardware required.

The aim of the invention is essentially to propose a particular solution to the problem which arises, which solution must make it possible to reduce the fingerprint-based identification process to a comparison pertaining to an amount of stored information which is sufficiently restricted for it to be possible to undertake this comparison under acceptable speed and cost conditions, while retaining a number of characteristic points which is high enough for the identification to remain reliable.

For these purposes, the invention proposes a method as mentioned in the preamble which is characterized in that the shape of at least one hand of all the persons of the population is sensed previously and the hand shape information is stored in said file in correlation with at least the respective fingerprint information, then in that at the time of identification of a person:

the shape of the hand of the person is sensed at the same time as the fingerprints of at least one of his/her fingers are sensed;

the shape information for his/her hand is compared with the hand shape information held in memory in the file for the entire population;

a group consisting of the hand shapes substantially identical to the sensed hand shape of the person to be identified is selected from said file; and

finally, the sensed fingerprints of the finger of the person to be identified are compared with the information stored in the file in correlation with respectively the hand shapes, of only the persons of the abovesaid selected group.

Certainly, various appropriate devices for sensing the geometry of a hand of an individual and for delivering a set of corresponding electrical signals which, digitized, may then be held in memory are already known.

However, hitherto it has never been proposed to combine, successively and in order, a sensing of the geometry of a hand and a sensing of the fingerprints of at least one finger of at least one hand for the purposes of identifying an individual.

The preselection of a group of persons on the basis of the geometry of the hand allows a considerable reduction in the exploratory domain within which the identification process based on fingerprint comparison will be undertaken. The process of comparing the geometry of a hand is a purely dimensional comparative process which can be carried out rapidly, and hence at less cost, even on a very sizeable population.

Optionally, by way of variant which may lead to increased speed, it is possible to envisage that groups of substantially identical hand shapes are constructed previously in the file and that at the time of identification of a person, the group of hand shapes substantially identical to the shape of the hand of the person to be identified is queried directly.

To fix matters, the equipment required by the implementation of the method in accordance with the invention may be reduced in a proportion from 10 to 1,or even from 20 to 1, as compared with the equipment to be envisaged in respect of identification solely through the sensing of fingerprints.

It is also advantageously possible to envisage that, after selection of the group of hand shapes substantially identical to those of the person to be identified and before comparing the fingerprints of the finger of the person to be identified with those of the persons of the abovesaid selected group, an additional selection is performed on the basis of at least one other selection criterion, which advantageously is a biometric criterion, in particular for example on the criterion of sex and/or type.

Thus, by virtue of the preselection performed according to the invention by sensing the geometry of a hand, optionally supplemented thereafter with additional preselections (according to sex which affords a halving, according to type, according to size, etc), it is eventually possible for the identification based on fingerprint comparison to pertain ultimately to only a small fraction of the population, it being possible to ensure this in a dependable, fast and economically acceptable manner.

The invention will be better understood on reading the detailed description which follows of certain preferred embodiments given merely by way of nonlimiting examples. In this description, reference will be made to the appended drawings in which:

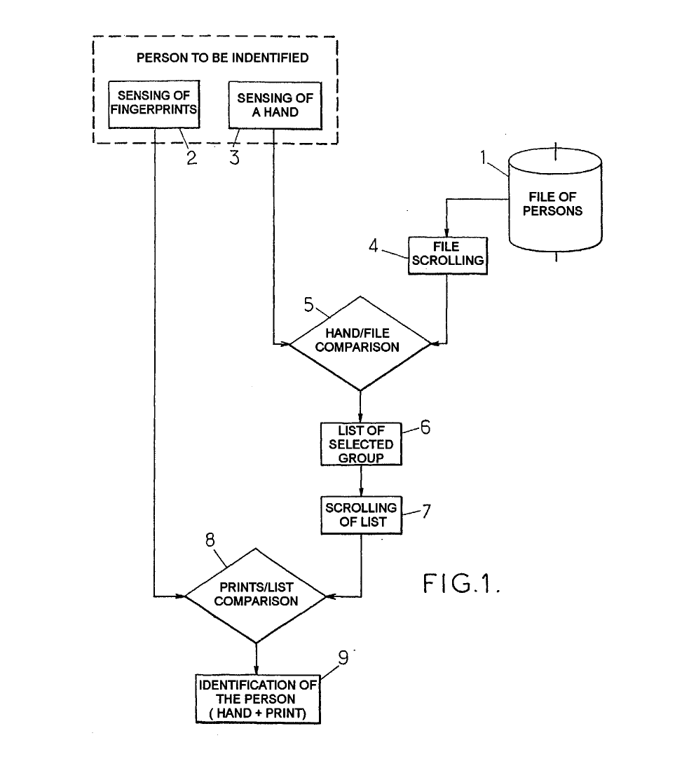

FIG. 1 is a schematic diagram illustrating the essential aspect of the method in accordance with the invention,

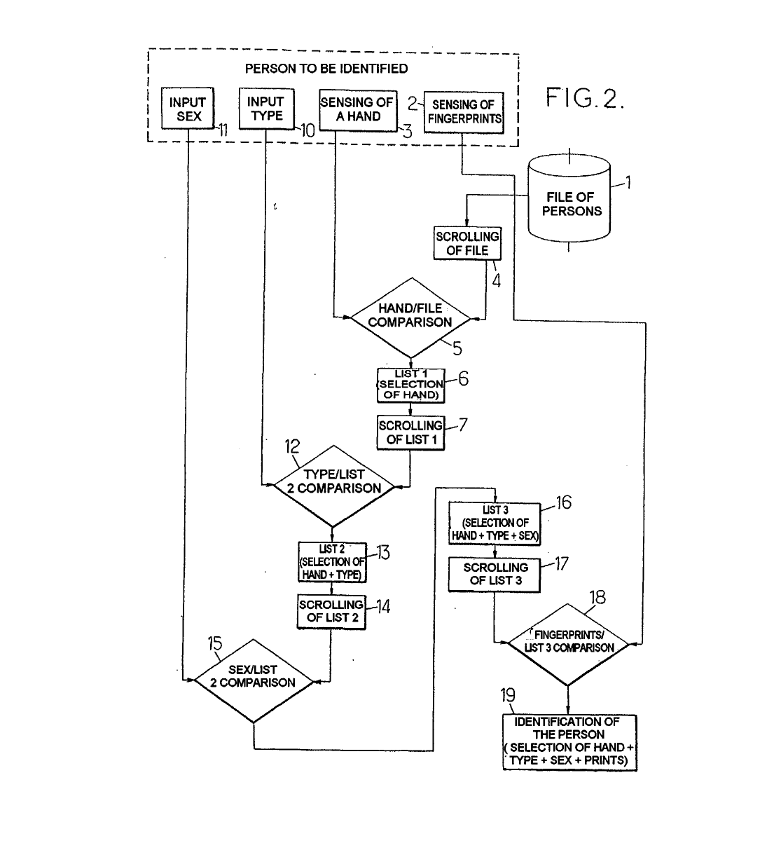

FIG. 2 is a schematic diagram illustrating a preferred embodiment of the method in accordance with the invention; and

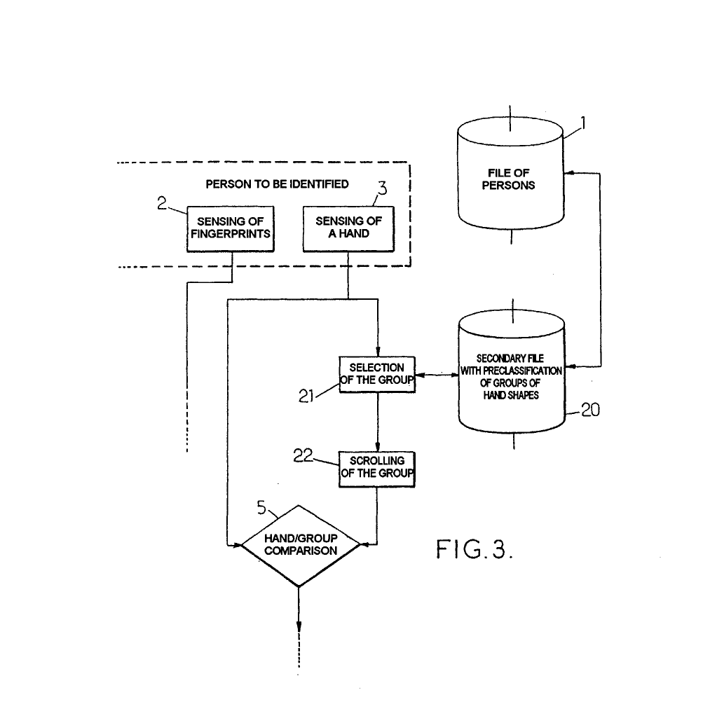

FIG. 3 is a schematic diagram illustrating a variant implementation of a part of the methods of FIGS. 1 and 2.

The means proposed by the invention must make it possible, for the purpose of identifying a person belonging to a very numerous population, to discard a large fraction of said population (for example 70 to 80% of the latter) before performing the procedure for identifying the person by comparing the fingerprints of at least one finger of at least one of his/her hands with the fingerprints stored in a central file accommodating all the identity data for the entire population.

The invention proposes to perform this preselection by charting the shape (geometry) of at least one hand of the person, given that the shape of a hand can be identified by a given number (for example a dozen) of dimensional measurements taken at characteristic points of the hand (for example lengths of the fingers, widths of the fingers at certain joints, thickness of the hand, etc) which are suitable for giving a three-dimensional image of the hand. Numerous methods and apparatuses are known in this regard.

Likewise, numerous methods and apparatuses are known which make it possible to chart the fingerprints of a finger, this sensing being performed in principle on one or more fingers of one hand or of both hands.

In an initial assessment phase, all the identification data for each person of a population is placed in memory in a central file 1 (see FIG. 1), which identification data, in addition to the customary data (name, first name, etc), also encompass the fingerprints of at least one finger (index finger for example) of at least one hand (right hand for example) as well as the dimensional data relating to the geometry of at least one of the hands (right hand for example).

Subsequently to check the identity of a person (police identity check, ballot, assessment, etc), physical identifying characteristics are sensed on the person, and especially his/her fingerprints (at 2) and the geometry of his/her hand (at 3) under the same conditions as during the initial assessment.

In a first step we begin by processing the information of the geometry of the hand sensed on the person to be identified. For this purpose, by scrolling at 4 the hand geometry data held in the central file 1 for all the persons of the population, and by comparison at 5 with those sensed on the person to be identified, a list of the group of persons of the population which possess a hand geometry substantially similar to that of the person to be identified is constructed at 6.

As indicated above, the domain to be explored has thus been restricted within very sizeable proportions, while selection based on the geometry of the hand, which pertains only to dimensional comparisons, is easy and fast to process with the powerful computing means currently available.

The method proceeds thereafter, while scrolling at 7 the list of groups of persons selected at 6, by individual comparison at 8 of the fingerprints sensed at 2 on the person to be identified with those of each of the persons of the group selected at 6.

The identification result for the person to be identified is finally obtained at 9.

Under these conditions, involving a numerous population (for example that of a country), the means proposed by the invention enable the complex and lengthy comparative identification via fingerprints to be made to pertain to a reduced group of persons and avoids the need to make it pertain to the entire population (which may for example reach several tens of millions of persons).

It will also be noted that, although this is not the essential aim of the invention, the reliability of identification of the person to be identified is incidentally increased on account of the fact that two identification criteria (geometry of the hand and fingerprints) are implemented rather than just one criterion (fingerprints). Certainly the degree of reliability of identification by fingerprints is very high. However, the identification which is carried out here is conducted computationally on the basis of the characteristics of the fingerprints: any risk of error due to the sensing of these characteristics and to the computerized processing may be compensated for through the implementation of the second selection criterion. Although in principle the method just set forth with regard to FIG. 1 leads to an implementational scheme which is economically acceptable in practice, it is however possible to push the preselection further on the basis of criteria which are very simple to process. For this purpose, criteria such as type, sex, etc may be invoked.

By way of example, in FIG. 2, it has been assumed that the preselection based on the geometry of the hand of the person to be identified has been supplemented with the input of two additional data consisting of its type (at 10) and his/her sex (at 11).

To this end, the type information, input at 10, relating to the person to be identified is compared at 12 with the list (list 1) of the group of persons selected from the file 1 on the basis of the geometry of their hand scrolled at 7.

This results in a second list (list 2) obtained at 13 of the persons selected among the population based on the twofold criterion of geometry of the hand and type.

The sex information, input at 11, relating to the person to be identified is thereafter compared at 15 with the list 2 scrolled at 14.

This then results in a third list (list 3) obtained at 16 of the persons selected among the population based on the threefold criterion of geometry of the hand, type and sex.

The fingerprint information can then be compared at 18 with the list 3 scrolled at 17, leading at 19 to the identification of the person.

The last comparison phase 18 then pertains only to a reduced number of persons, thereby rendering the comparative processing of the fingerprints executable with considerably reduced computer hardware (reduction by 10 or 20 for example).

Here again, in addition to the speeding up of the identification processing and the reduction in investment in hardware, the multiplying of the preselection criteria preceding the identification by fingerprints substantially increases the reliability of the method.

By way of variant, in order to avoid the comparative processing of the geometry of the hand of the person to be identified from being performed by systematic comparison with the entire data of the population, it is possible to envisage, in order to save time, the creation, from the main file 1, of a secondary file 20 in which groups bringing together each of the persons possessing substantially the same hand geometry have been created.

In this case, as illustrated in FIG. 3, on the basis of the hand geometry data charted at 3 on the person to be identified, the secondary file 20 is searched at 21 for the corresponding dimensional group and it is then by scrolling at 22 of this preconstructed group alone that the previously envisaged comparison is performed at 5.

Of course, numerous alterations may be made to the method set forth hereinabove without departing from the scope of the invention.

1. A method for identifying a person among a population, by sensing the fingerprints of at least one finger of the hand of said person and comparing these fingerprints with fingerprint information from all the persons of said population previously stored in a file (1), characterized in that the shape of at least one hand of all the persons of the population is sensed previously and the hand shape information is stored in said file (1) in correlation with at least the respective fingerprint information, then in that at the time of identification of a person:
the shape of the hand of the person is sensed (3) at the same time as the fingerprints of at least one of his/her fingers are sensed (2);
the shape information for his/her hand is compared (5) with the hand shape information held in memory in the file (1) for the entire population;
a group consisting of the hand shapes substantially identical to the sensed hand shape of the person to be identified is selected (6) from said file; and
finally, the sensed fingerprints of the finger of the person to be identified are compared (8) with the information stored in the file in correlation with respectively the hand shapes, of only the persons of the abovesaid selected group.

2. The method as claimed in claim 1, characterized in that, after selection (6) of the group of hand shapes substantially identical to those of the person to be identified and before comparing the fingerprints of the finger of the person to be identified with those of the persons of the abovesaid selected group, an additional selection is performed on the basis of at least one other selection criterion.

3. The method as claimed in claim 2, characterized in that the additional selection criterion applied to the persons of the selected group of hand shapes is a biometric criterion such as sex and/or type.

4. The method as claimed in any one of the preceding claims, characterized in that groups of substantially identical hand shapes are constructed (20) previously in the file (1) and in that at the time of identification of a person, the group of hand shapes substantially identical to the shape of the hand of the person to be identified is queried (21) directly.
